(19) United States
(12) Patent Application Publication
(10) Pub. No.: US 2006/0015026 A1
(43) Pub. Date: Jan. 19, 2006

(54) Porous coatings on electrodes for biomedical implants

(76) Inventors: David A. Glocker, West Henrietta, NY (US); Mark M. Romach, Spencerport, NY (US)

Correspondence Address: Mauri Aven Sankus, Esq.;Jaeckle Fleischmann & Mugel, LLP, 190 Linden Oaks, Rochester, NY, 14625-2812, US

(21) Appl. No.: 11/180,183

(22) Filed: Jul. 13, 2005

Related U.S. Application Data
(60) Provisional application No. 60/587,408, filed on Jul. 13, 2004.

(51) Int. Cl. A61B 5/04; A61N 1/36

(52) U.S. Cl. 600/373; 607/9; 600/374

(57) ABSTRACT

An implantable electrode has a biomedically compatible, microscopically rough, metal coating that creates a high double-layer capacitance. The coating is applied to the implant by physical vapor deposition. The coating preferably is applied via a generally oblique coating flux or a low energy coating flux. In some embodiments, the coating has pores. The pores can contain a drug, which can diffuse over a period of time. The coating may be partially nonporous to protect the implant from corrosion.

### CROSS REFERENCE TO RELATED APPLICATION

This application claims the benefit of U.S. provisional application No. 60/587,408 filed Jul. 13, 2004, the entire disclosure of which is incorporated herein by reference in its entirety for any and all purposes.

### TECHNICAL FIELD

The present invention generally relates to medical devices and, more particularly, to implantable medical devices having electrodes and methods of making implantable medical devices having electrodes.

### BACKGROUND

Sensing and delivering low voltage signals within the human body has become an extremely important technology. Examples include cardiac pacemakers for regulating a patient's heartbeat and devices that deliver impulses to nerves to manage pain.

A cardiac pacemaker consists of a pulse generator containing a source of power and leads that carry low voltage signals to and from the heart. The generator includes circuitry that produces electrical impulses causing the heart to beat properly and it may also sense and respond to small voltages produced by the heart. The electrodes that stimulate the heart muscles are known as effectors and those that sense signals from the heart are known as sensors. The generator is often placed near the patient's collarbone and the electrical leads in those cases may pass into the heart through a blood vessel, such as the left subclavian vein. The proximal end of an effector or sensor lead is connected to the generator and the distal end is implanted in the patient's heart.

The nature of the electrical contact between the distal end of such leads and the myocardial tissue of the heart is extremely important and a great deal of work has been done to optimize the design of pacemaker leads. For example, it is widely known that a high double layer capacitance, i.e. in the range of approximately 10 to 100 milli Farads/cm² between the end of the lead and the surrounding tissue is very important. A high capacitance for an effector electrode desirably reduces the polarization rise associated with stimulation pulses. And a high capacitance for a sensor electrode will desirably lower the source impedance and reduce the signal attenuation in the amplifier of the generator. Finally, the electrode material must be biologically inert and stable over time.

One means of creating a high double layer capacitance is to use an electrode having a large surface area. In order to increase the surface area while keeping the overall dimensions of the electrode small, one prior art method is to use a sintered powder of an electrical conductor having a very porous structure. Examples are sintered powders of tantalum, titanium, niobium, zirconium, platinum, glassy carbon and cobalt-chromium alloys, which are the subjects of U.S. Pat. Nos. 4,440,178; 5,282,844; and 4,934,881. Also described in the prior art are porous non-conducting substrates in conjunction with conducting electrodes, which are the subjects of U.S. Pat. Nos. 4,784,161 and 4,844,099. While these prior art inventions raise the capacitance of electrodes, they generally do not raise the capacitance sufficiently.

An alternative method known in the prior art to produce a large surface area is to apply a porous coating to the distal end of the implanted electrode. Desired double layer capacitances for porous coatings are in the range of 10 to 100 milli Farads/cm², which is significantly greater than the double layer capacitances for sintered powder electrodes described in the previous paragraph. The preferred method for applying such coatings is physical vapor deposition. Prior art coating materials include the carbides, nitrides or carbonitrides of metals in the group containing titanium, vanadium, zirconium, niobium, molybdenum, hafnium, tantalum or tungsten. These compounds can be produced by physical vapor deposition under coating conditions that result in very rough, porous microstructures. This is the subject of U.S. Pat. Nos. 4,603,704; 4,611,604; 5,991,667 and published application US 2001/0032005 A1. Of these materials, titanium nitride is almost exclusively used in these applications. However, the long term chemical stability of titanium nitride is a concern.

Published application US 2001/0032005 A1 addresses the titanium nitride stability issue by depositing a layer of iridium or iridium oxide on top of the porous titanium nitride to protect it. In that patent application the inventors explain that iridium and iridium oxide are desirable from the standpoint of tissue compatibility, but neither iridium metal nor iridium oxide can be produced with the desired microstructure.

Moreover, the myocardial tissue should bond strongly to the electrode tip to prevent it from moving once it is in place. Therefore, the distal end of the implanted electrode must not only have the required electrical properties, but the coating should adhere tenaciously to the conducting tip.

Therefore, what is needed is an implantable electrode that has a capacitance in the range of approximately 10 to 100 milli Farads/cm² and is chemically stable. In addition, the electrode is preferably simple to manufacture.

### SUMMARY

The present invention is directed towards an implantable electrode comprising a body and a biomedically compatible, microscopically rough, metal coating applied to at least a portion of the body via physical vapor deposition.

The electrode has a capacitance between approximately 10 and 1000 milli Farads/cm². The coating preferably has surface features having a size between 10 nm and 1000 nm. These features may vary in size. The coating can comprise one of the group of tantalum, titanium, molybdenum, vanadium, hafnium, niobium, tungsten, platinum and zirconium. Preferably, the coating has a thickness between 1 and 100 micrometers. In the preferred embodiment, the coating has pores. A drug may reside within the pores.

The electrode can have a second coating. The second coating can be applied directly to the electrode and the microscopically rough, preferably porous, coating can be applied to the second coating. Optionally, the second coating protects the electrode from corrosion and is nonporous.

The physical vapor deposition method comprises one of the group of sputtering, cathodic arc deposition or thermal evaporation. The coating preferably is applied to the electrode via one of a generally oblique coating flux or a low energy coating flux.

A process for making a high capacitance portion of an implantable electrode comprises the steps of:
maintaining a background pressure of gas in a sputter coating system containing at least one sputter target;
applying a voltage to the target to cause sputtering; and
sputtering for a period of time to produce a microscopically rough, metal coating on a portion of the electrode.

An implantable medical device comprises:
a body; and
a biomedically compatible, microscopically rough metal coating applied to at least a portion of the body via physical vapor deposition.

### BRIEF DESCRIPTION OF DRAWINGS

These and other features, aspects and advantages of the present invention will become better understood with regard to the following description, appended claims, and accompanying drawings where:

FIG. 1 is a partial front view of an electrode in accordance with the current invention;

FIG. 2 is a top view of a target surrounding substrates;

FIG. 3 is a side cross-sectional view of the target surrounding substrates of FIG. 2

FIG. 4 is a side cross-sectional view of the target surrounding substrates in position C of FIG. 3 with a plate above the substrates;

FIG. 5 is a top view of a target surrounding substrates in another configuration;

FIG. 6 is a side cross-sectional view of the target surrounding substrates of FIG. 5;

FIG. 7 shows a scanning electron micrograph of the surface of a Ta coating applied to a polished stainless steel surface;

FIG. 8 is a side elevation view of substrates positioned beside a planar target at a high angle of incidence; and

FIG. 9 shows an atomic force microscopy image of a Ta coating made according to another preferred embodiment of the present invention and applied to a polished nickel titanium alloy substrate.

### DESCRIPTION

The present invention is directed towards an implantable electrode having a microscopically rough outer coating that has a capacitance in the range of approximately 10 to 100 milli Farads/cm². The coating of the preferred embodiment also adheres well to the body of the electrode and improves the adhesion of the electrode to natural tissue. The preferred electrodes are extremely stable and are ideal for use in cardiac pacemakers.

By microscopically rough, we mean having surface features, including but not limited to, pores, bumps, hollows or combinations thereof, on the order of 10's to 100's of nanometers in size. These features can be seen using a scanning electron microscope. Preferably, however, the surface features include pores, as a porous coating has a higher capacitance that a non-porous coating.

The coating preferably is applied by physical vapor deposition processes, such as sputtering, cathodic arc or thermal evaporation. In some cases the coatings can also be infused with materials intended for a variety of purposes, such as to prevent inflammation or promote tissue growth.

Tantalum is biomedically compatible and corrosion resistant, making it an attractive material for the microscopically rough coatings in this application, although other materials may be used, such as, but not limited to, titanium, vanadium, molybdenum, niobium, hafnium, zirconium, tungsten and platinum and combinations thereof. We have also found that it is possible to control the crystal structure of the Ta in a reliable way.

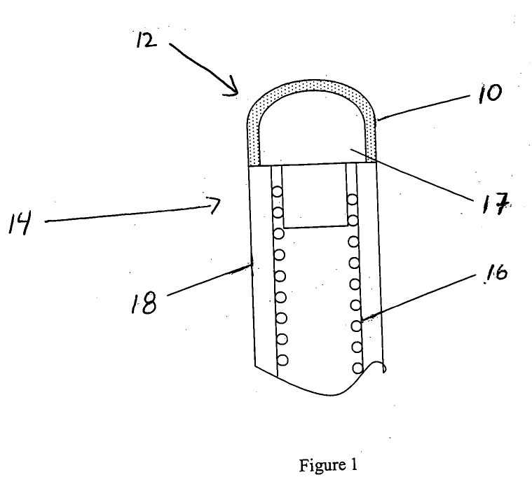

FIG. 1 schematically shows the construction of one embodiment of the distal end 12 of a lead 14 made in accordance with the present invention. The lead 14 itself is typically made of a flexible conductor 16, which is covered with an insulating sheath 18. The conductor 16 may be fine wire that is wound in a helical shape to increase the overall flexibility of the structure. The proximal end (not shown) of the lead 14 is connected to a generator (not shown) and either provides a sensing signal in the case of a sensor lead or delivers a pacing signal in the case of an effector lead. This arrangement is described in U.S. Pat. No. 4,603,704, for example. A conducting tip 17 in electrical contact with the lead 14 is coated with a porous coating 10 comprising tantalum or another suitable metal.

It is well known in the art of physical vapor deposition that low homologous coating temperatures (the ratio of the substrate temperature to the melting point of the coating material in degrees Kelvin) often result in microscopically rough, porous coatings. However, poor coating adhesion also often results from these coating conditions. Nevertheless, we have unexpectedly found that rough, porous coatings deposited under the correct conditions are able to adhere to the types of materials used in biomedical electrodes without unacceptable flaking.

A large number of experiments were done to examine the influence of the deposition conditions and system geometry on the structure of the resulting coatings. In all cases the electrode materials, sometimes referred to as “substrates” herein, were cleaned with a warm aqueous cleaner in an ultrasonic bath. Crest 270 Cleaner (Crest Ultrasonics, Inc.) diluted to 0.5 pounds per gallon of water was used at a temperature of 55 C. This ultrasonic detergent cleaning was done for 10 minutes. The substrates were then rinsed for 2 minutes in ultrasonically agitated tap water and 2 minutes in ultrasonically agitated de-ionized water. They were then blown dry with nitrogen and further dried with hot air. The manner in which the substrates were cleaned was found to be very important. When the substrates were cleaned ultrasonically in acetone and isopropyl alcohol, a residue could be seen on the substrates that resulted in poor adhesion.

In addition to conventional cleaning, it is possible to use plasma cleaning as an integral part of the coating process. In that case, an initial high voltage can be applied to the substrates in order to sputter clean them and remove any residual contamination. The initial high voltage preferably is between approximately 100 and 600 volts and is preferably applied for about 20 minutes. This cleaning can be done with the deposition source off or it can be carried out during the initial stages of deposition. Times for such cleaning can be from less than a minute to several minutes. A second lower voltage can be applied, preferably for a period of time between about 1 and 5 hours.

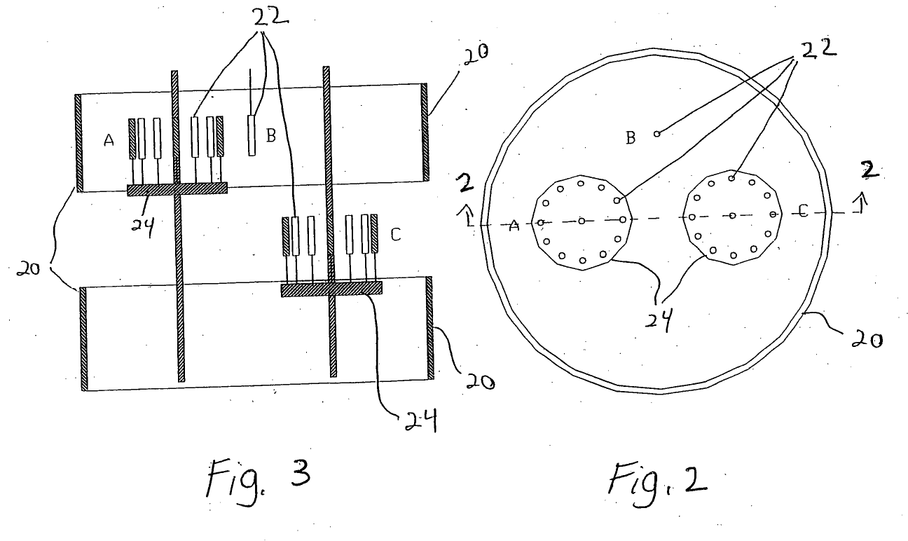

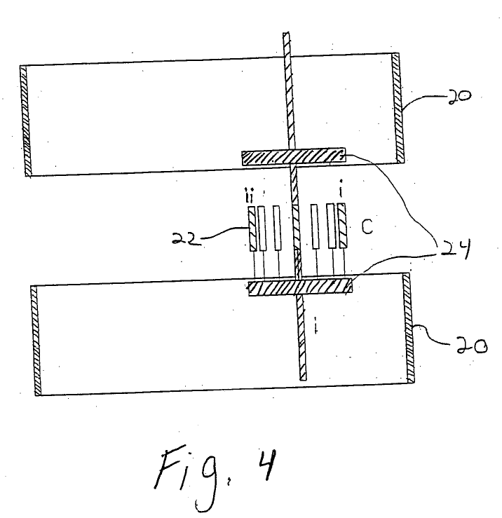

Two different unbalanced cylindrical magnetron sputtering systems, as described in U.S. Pat. No. 6,497,803, which is incorporated herein by reference, were used to deposit the coatings. FIGS. 2 and 3 illustrate the setup for system 1. System 1 had targets 20, each 34 cm in diameter and 10 cm high, separated by 10 cm. System 2 was similar to system 1 but only used the top target shown in FIGS. 2 and 3, which was 19 cm in diameter. Preferably Ar, Kr or Xe was used as the sputtering gas, sometimes in mixtures with other gases. In general, the targets can be cylinders or plates or any other form known in the art. They were driven with either DC power or AC power. Other devices well known to those in the art, such as vacuum pumps, power supplies, gas flow meters, pressure measuring equipment and the like, are omitted from FIGS. 2 and 3 for clarity.

Preferably, two independent power supplies are used in the case of DC power and a single power supply connected to both targets is used in the case of AC power in a manner well known to those skilled in the art. The voltage can be applied continuously or in pulses or in any other manner known in the art. Preferably, the voltage produces a deposition rate of one to 5 microns per hour.

The sputtering targets 20 were preconditioned at the process power and pressure for approximately 10 minutes prior to starting the depositions. During this step a shutter isolated the substrates 22 from the targets 20. Importantly, this preconditioning process heated the shutter and caused the temperature of the substrates 22 to rise. This preheating allowed the substrates 22 to further degas and approach the actual temperature of the coating step. The substrates 22 were not directly heated or cooled in any way during deposition and their time-temperature history was determined entirely by the coating process. During sputtering the substrate temperature preferably remains between 150 and 450 degrees Celsius. This is a very low homologous temperature for materials such as Ta, Ti, Mo and Nb. After opening the shutter, the coating time was adjusted so that a coating thickness of approximately 10 microns resulted. At a power of 4 kW the time for Ta was 2 hours and 15 minutes and at a power of 2 kW the time was 4 hours and 30 minutes. For clarity, these are the time/power combinations that achieve a 10 micron coating thickness for Ta. In some of the examples below, the coating times vary from those given above. When this is the case, the coating thickness varies also.

### Example 1

Electropolished nickel-titanium alloy substrates 22 were placed at three positions in System 1, as shown in FIGS. 2 and 3:

Position A—The substrates 22 were held on a 10 cm diameter plate 24 that rotated about a vertical axis, which axis was approximately 7 cm from the cathode centerline. The vertical position of the substrates 22 was in the center of the upper cathode. Finally, each substrate was periodically rotated about its own axis by a small “kicker” in a manner well known in the art.

Position B—The substrates 22 were suspended from a rotating axis that was approximately 7 cm from the chamber centerline. The vertical position of the substrates 22 was in the center of the upper cathode.

Position C—The substrates 22 were on a 10 cm diameter plate 24 that rotated about a vertical axis, which axis was approximately 7 cm from the cathode centerline, as in position A. However, the vertical location of the substrates 22 in position C was in the center of the chamber midway between the upper and lower cathodes. Finally, each substrate was periodically rotated about its own axis with a “kicker.”

The targets 20 were Ta and were each driven at a DC power of 2 kW. A bias of −150V was applied to the substrates 22 during the coating. The sputtering pressure was 3.4 mTorr and the sputtering gas was Kr. The coating time was 2 hours and 15 minutes, resulting in a coating thickness of about 10 microns.

There was a marked difference in the appearance of the substrates 22 at the three positions. Those in positions A and, B were shiny and metallic, while the substrate in position C had a dull, matte metallic appearance.

### Example 2

To further explore the influence of the substrate position in the chamber on the appearance of the coating, an experiment was done in which only the top Ta target was operating at a power of 2 kW in System 1. The sputtering pressure was 3.4 mTorr, the sputtering gas was Kr and the coating time was 3 hours and 20 minutes. Nickel titanium alloy substrates 22 were located in positions B and C shown in FIGS. 2 and 3.

The substrate in position B was shiny and metallic looking. The substrate in position C was somewhat shiny on the top, but was black at the bottom. It is well known that a black appearance can result from a surface with microscopic features on the order of hundreds of nanometers because of the scattering and absorption of visible light.

The adhesion of the coatings was tested using 3M Scotch Brand tape. The tape was pressed into the substrates 22 and pulled away. There was significant removal of the coating from the substrate in position B, but only one small spot of removal at the top of the substrate in position C and no removal from the lower portion with the black appearance.

In this experiment the substrate in position C received a generally more oblique and lower energy coating flux than the substrate in position B. By an oblique coating flux we mean that the majority of the depositing atoms arrive in directions that are not generally perpendicular to the surface being coated. Some of the atoms arriving at the surfaces of the substrate in position C from the upper target will have done so without losing significant energy or directionality because of collisions with the background sputter gas. Those atoms, most of which will come from portions of the target close to the substrate as seen in FIG. 3, will create an oblique coating flux. Other atoms will undergo several collisions with the background gas and lose energy and directionality before arriving at the substrate surfaces. Those atoms, which will generally come from portions of the target at greater distances, will form a low average energy coating flux.

Westwood has calculated (“Calculation of deposition rates in diode sputtering systems,” W. D. Westwood, Journal of Vacuum Science and Technology, Vol. 15 page 1 (1978)) that the average distance a Ta atom goes in Ar at 3.4 mTorr before its energy is reduced to that of the background gas is between about 15 and 30 cm. (The distance would be somewhat less in Kr and the exact value depends on the initial energy of the Ta atom.) Because our cylindrical targets 20 have an inside diameter of approximately 34 cm, substrates 22 placed in the planes of the targets (positions A and B) receive a greater number of high energy, normal incidence atoms and those placed between the targets 20 (position C) receive a greater number of low energy and/or oblique incidence atoms.

The geometry of the cylindrical magnetron arrangement shown in FIGS. 2 and 3 assures that atoms arriving at the surface of substrates 22 placed in position C will do so either at relatively oblique angles or with relatively low energy. Referring to FIG. 3, when the substrates 22 are close to the targets 20 where the arriving Ta atoms have lost little energy, the atoms arrive at oblique angles. And when the substrates 22 move closer to the center of the chamber where the arrival angles are less oblique, they are farther from the target surface so that the arriving Ta atoms have lost more energy through gas collisions.

Typically, sputtered atoms leave the target surface with average kinetic energies of several electron volts (eV). As described by Westwood, after several collisions with the background gas the sputtered atoms lose most of their kinetic energy. By low energy, we are referring to sputtered atoms that have average energies of approximately 1 eV or less. Westwood's calculations can be used to estimate the target to substrate spacing required to achieve this low average energy for a given sputtering pressure. Furthermore, it is well known to those skilled in the art that atoms deposited by evaporation have average energies below approximately one eV when they leave the evaporation source. Therefore, scattering from the gas in the chamber is not required to produce a low energy coating flux in the case of evaporated coatings.

It is widely known in the art that when the atoms in a PVD process arrive with low energies or at oblique angles to the substrate surface, the result is a coating that can have a rougher surface and lower density than a coating made up of atoms arriving at generally normal incidence or with higher energies. As discussed earlier, the black appearance of the coating in position C may be the result of coating roughness on the order of tens to hundreds of nanometers in size. Those skilled in the art will recognize that the rough, porous coatings we are describing are those sometimes called Zone 1 coatings for sputtered and evaporated materials (see, for example, “High Rate Thick Film Growth ” by John Thornton, Ann. Rev. Mater. Sci., 1977, 239-260). Zone 1 coatings are characterized by columnar structures with voids between the columns. Deposition conditions that produce such coatings typically lead to poor adhesion. Surprisingly, we have found excellent adhesion in such coatings made by our methods.

### Example 3

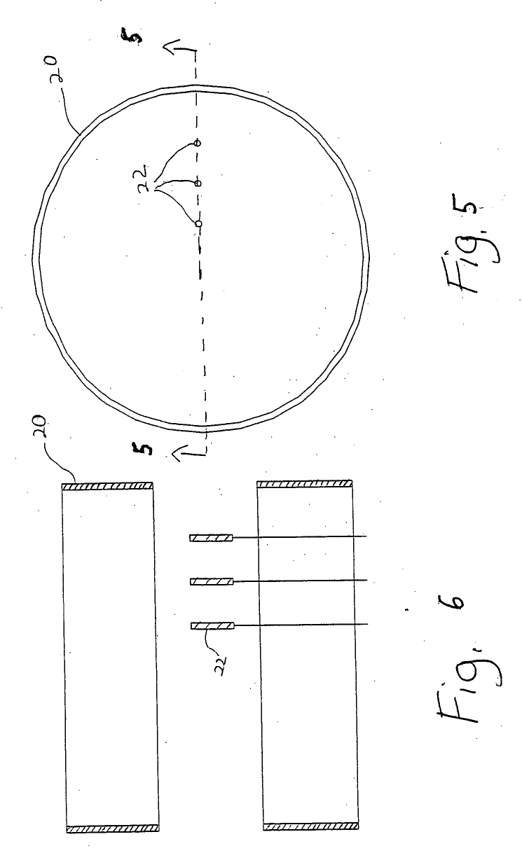

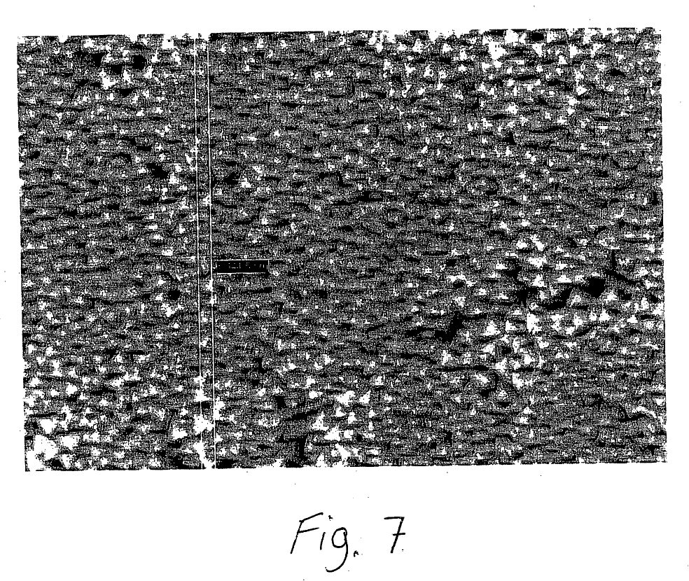

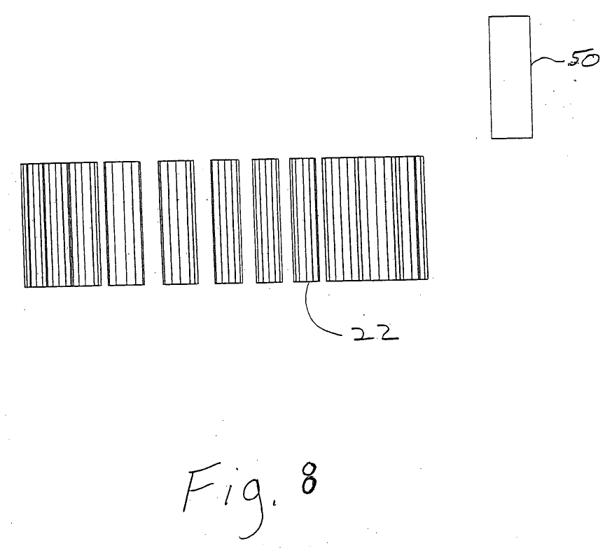

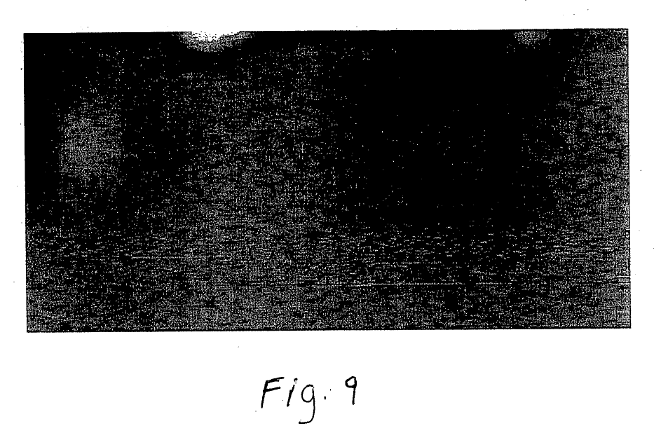

Further evidence of the importance of the coating geometry and sputtering conditions is seen in the following experiment, illustrated in FIGS. 3 and 4. A number of Ta coatings were done on nickel titanium alloy substrates 22 in System 1 using Kr at a pressure of 3.4 mTorr, a DC power of 1 kW on each target and a bias of −50 V and the plate 24 shown in FIG. 3 position C. As before, the substrates 22 were rotating about the vertical rod as well as about their own axes. In order to increase the effect of position in this experiment, 10 cm long substrates 22 were used. The coatings made this way were matte black at the bottom but had a slightly shinier appearance at the top. In contrast, when coatings were done on substrates 22 under identical conditions, except that a second plate 24 was placed above the substrates as shown in FIG. 4, the substrates were a uniform black from bottom to top.

The non-uniformity in appearance that resulted with the fixturing shown in FIG. 3 is further evidence that the coating structure depends on the details of how the substrates 22 and sputter targets 20 are positioned relative to one another. As discussed earlier, when the substrates 22 are in position Ci in FIG. 4, they receive very oblique incidence material from portions of the targets 20 that are close, while the coating material that arrives from other portions of the targets has to travel farther. Therefore, all of the coating flux has arrived at oblique incidence or has traveled a considerable distance and has lost energy and directionality through collisions with the sputtering gas. When the substrates 22 are in position Cii in FIG. 4, however, they receive a somewhat less oblique coating from all directions. In the configuration shown in FIG. 3, however, the bottoms of the substrates 22 are shielded from the more direct flux from the bottom target by the plate 24 that holds them, but the tops of the substrates 22 are not similarly shielded from the more direct flux coming from the top target. By adding the plate 24 above the substrates 22 as well, as shown in FIG. 4, the more direct coating flux is shielded at all points on the substrates and the coating material either arrives at relatively oblique incidence or after scattering from the background gas and losing energy and directionality. The plate 24 above the substrates 22 restores symmetry and the coatings on the substrates become uniformly black.

### Example 4

Other methods of positioning and moving the substrates 22 within the chamber can also produce results similar to those described above and are within the scope of the invention. In another experiment three nickel titanium alloy substrates 22 were located in System 1 as shown in FIGS. 5 and 6. FIG. 5 is a top view of the substrate locations and FIG. 6 is a cross-sectional view of the same arrangement. All three were held fixed at their positions within the chamber and were rotated about their individual axes during the coating run. The innermost substrate was 3 cm from the cathode centerline, the middle substrate was 7 cm from the cathode centerline and the outermost substrate was 11 cm from the cathode centerline. The Ta deposition was done at a DC power of 1 kW on each target, a Kr pressure of 3.4 mTorr and with the substrates 22 biased at −50 V. All three substrates 22 had a matte black appearance and none of the coating could be removed from any of the substrates using the tape test. Therefore, substrates 22 placed at virtually any radial position within the cathodes and rotating about their individual axes will receive a satisfactory coating, provided they are located between the targets in the axial direction.

An alternative to oblique incidence coatings or large target to substrate distances in order to reduce the energy of the arriving atoms through collisions is to raise the pressure of the sputtering gas. It is widely known in the art that high sputtering pressures lead to less dense coatings with microscopically rough surfaces. However, we have found that this approach can produce less desirable results.

Sputtering takes place under conditions of continuous gas flow. That is, the sputtering gas is brought into the chamber at a constant rate and is removed from the chamber at the same rate, resulting in a fixed pressure and continuous purging of the gas in the chamber. This flow is needed to remove unwanted gases, such as water vapor, that evolve from the system during coating. These unwanted gases can become incorporated in the growing coating and affect its properties.

The high vacuum pumps used in sputtering, such as diffusion pumps, turbomolecular pumps and cryogenic pumps, are limited with respect to the pressure that they can tolerate at their openings. Therefore, it is well known that in order to achieve high sputtering pressures it is necessary to “throttle” such pumps, or place a restriction in the pump opening that permits the chamber pressure to be significantly higher than the pressure at the pump. Such “throttling” necessarily reduces the flow of gas through the chamber, or gas throughput. Surprisingly, we have found that adherent coatings depend on having high gas throughputs and pumping speeds, which is only practical at relatively low sputtering pressures. Our results indicate that during sputtering, preferably the gas throughput is between approximately 1 and 10 Torr-liters per second.

### Example 5

In one experiment, a single target 20 of System 2 having an inside diameter of 19 cm and length of 10 cm was used to coat an electropolished nickel-titanium alloy substrate 22 with Ta at a sputtering pressure of 30 mTorr in Ar. In order to achieve this pressure, it was necessary to throttle the turbomolecular high vacuum pump on the vacuum system. The Ar flow during this coating was 0.63 Torr-liters per second, corresponding to a throttled pumping speed of 21 liters per second. The substrate 22 was placed in the center of the target 20, approximately 9 cm from the target surface. The DC sputtering power to the target was 200 W. According to Westwood's calculations, the average distance a Ta atom travels in Ar at 30 mTorr before reaching thermal velocities is between 1.7 and 3.4 cm, depending on its initial energy. Therefore, these coating conditions should result in a very low-density and microscopically rough coating. The black appearance of the coated substrate confirmed that this was the case. However, the coating had very poor adhesion.

### Example 6

In another experiment, Ta coatings were done on nickel titanium alloy substrates 22 in the C position using System 1 as shown in FIG. 3. The sputtering gas was Kr at a pressure of 3.4 mTorr. A DC power of 1 kW on each target 20 was used together with a substrate bias of −50 V. The Kr flow was 28 standard cubic centimeters per minute, or 0.36 Torr-liters per second. At a pressure of 3.4 mTorr this corresponds to a throttled pumping speed of 104 liters per second during the process. The resulting black coatings had adhesion failure in several locations when using the adhesive tape test.

The position of the pump throttle was then changed and the Kr flow was increased to 200 standard cubic centimeters per minute or 2.53 Torr-liters per second. Coatings were done on substrates 22 in the C position at the same power, pressure and bias levels as before. The only difference was that the throttled pumping speed during this process was 744 liters per second. In this case there was no removal of the coating from the substrate using the tape test.

Based on the foregoing results, we conclude that adequate adhesion may not result at low gas throughputs, which are usually necessary to achieve high sputtering pressures. The sputtering pressure and system geometry must be chosen together so that the coating flux arrives at the substrate surface either at high angles of incidence or after the sputtered atoms have traveled a sufficient distance from the target 20 to reduce their energies significantly.

### Example 7

In order to test the usefulness of these coatings on other materials and examine their structure, electropolished stainless steel substrates 22 were located in position C in System 1 as shown in FIG. 3. The system was operated at a sputtering power of 1 kW on each Ta target 20, a bias of −50V applied to the substrates 22 and a pressure of 3.4 mTorr at a throughput of 2.5 Torr-liters per second. The deposition time was 2 hours and 15 minutes.

The coatings were black. The adhesion of the coatings to the substrates 22 was assessed using the tape test and several attempts failed to remove the coating. Moreover, the tape stuck much more tenaciously to the coated substrates 22 than to similar uncoated substrates. This indicates the presence of a rough, porous structure on the surface.

FIG. 7 shows a scanning electron micrograph of the Ta coating on the stainless steel substrates 22. The substrates 22 were extremely smooth and the surface roughness and open structure that result from the coating are clearly visible. Many of the surface features have sizes of less than a micron. X-ray diffraction scans of this coating showed that it consisted almost entirely of the body centered cubic phase of Ta. While the geometry of a cylindrical magnetron makes oblique incidence coatings possible in an efficient way, as we have shown, the same results can be accomplished using planar targets as well. In the case of planar targets, the requirement is to place the substrates 22 far enough from the target surface(s) that a large target-to-substrate distance is achieved. Alternatively, the substrates 22 could be placed to the side of a planar target 50 so that the material arrives at high incidence angles. This configuration is illustrated in FIG. 8. Of course, the substrate positions shown in the case of planar targets make inefficient use of the coating material and greatly reduce the deposition rate, which are undesirable from a manufacturing standpoint. Nevertheless, FIG. 8 illustrates how the inventive method could be used with geometries other than cylindrical magnetrons.

### Example 8

We have also discovered that the initial coating conditions can influence the microstructure and crystalline phase of our coatings while preserving excellent adhesion. In one experiment, substrates 22 were loaded in Position C in System lusing the setup shown in FIG. 3 with 34 cm diameter targets 20. With the shutter closed, the two Ta targets 20 were operated at 2 kW (1 kW each) at a Kr pressure of 3.6 mT and a Kr flow of 2.53 Torr-liters per second. After five minutes, and with the shutter still closed, a voltage of −200 V was applied to the substrates 22 in order to plasma clean them. The shutter was opened after five additional minutes and the coating was begun with a −200 V bias still applied to the substrates 22. These conditions were maintained for two minutes, at which time the voltage on the substrates 22 was reduced to −50 V and the coating was deposited under these conditions for three hours. There was no flaking evident on these substrates 22.

Except for the initial five minutes of plasma cleaning and two minutes of −200 V bias sputtering, the conditions in the example above were the same as those used in Example 7 that produced the structure shown in FIG. 7 and the bcc crystalline phase. FIG. 9 is an atomic force microscope image of the resulting coating showing that the microstructure is changed by the initial conditions. While the features in FIGS. 7 and 9 are similar and both are microscopically rough, porous coatings, a close analysis shows that the structures in FIG. 7 are approximately 100 to 200 nm in size, while those in FIG. 9 are about twice as large. Moreover, the X-ray diffraction pattern shows that the crystalline phase of this coating shown in FIG. 9 was primarily tetragonal, with some bcc present.

Examples 7 and 8 show that a variety of coating conditions can be used to make the microscopically rough, porous structures we are describing. Moreover, they also show that it is possible to control the microstructure and crystalline phase through the proper choice of coating conditions.

The combination of a very porous coating and excellent adhesion is very surprising. Oblique coating fluxes, thermalized coating atoms and low homologous temperatures are known to produce open, columnar coating structures and microscopically rough surfaces. However, such coatings typically have very poor adhesion. We have found conditions that produce such structures along with excellent adhesion.

An open, porous structure may have other advantages for implantable medical devices as well. For example, the microvoids in the coating would permit the incorporation of drugs or other materials that could diffuse out over time. Examples are superoxide dismutuse to prevent inflammation or proteins to promote tissue growth, or other materials that aid in the healing or growth process. In the art, drug-eluting coatings on substrates are presently made using polymeric materials. A porous inorganic coating would allow drug-eluting substrates to be made without polymeric overcoats.

The process described in the present invention provides a simple, inexpensive method for producing surfaces on implantable electrodes produce a high double layer capacitance. In addition to tantalum, other materials that could be used include titanium, molybdenum, zirconium and other biocompatible elements. Moreover, it is possible to alter the surface layers of such coatings by anodizing or nitriding them or to deposit the oxides or nitrides of metals directly.

It is also possible to vary the conditions to produce a coating whose properties change throughout the thickness. For example, the first part of the coating could be applied. under conditions that produce a fully dense coating. Then the conditions could be changed to those that produce a porous open structure. Such a coating could provide corrosion protection for the electrode by virtue of the initial dense layer and good adhesion to bone through the microscopically rough layer above. In addition, drugs that diffuse over time can reside in the pores. Similarly, a nonporous coating can be applied to protect the substrate from corrosion. Then, an outer porous layer can be applied that easily bonds with animal tissue.

Although the present invention has been described in considerable detail with reference to certain preferred versions thereof, other versions are possible. For example, a substrate can be coated with a layer of a first material and a layer of a second, porous material. In another example, the microscopically rough features can be bumps instead of pores, the features can be a combination of bumps and pores. Therefore, the spirit and scope of the appended claims should not be limited to the description of the preferred versions contained herein.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function should not be interpreted as a “means” or “step” clause as specified in 35 U.S.C. §112.

We claim:

1. A implantable electrode comprising:
a body; and
a biomedically compatible, microscopically rough, metal coating applied to at least a portion of the body via physical vapor deposition.

2. The electrode of claim 1 wherein the electrode has a capacitance between approximately 10 and 1000 milli Farads/cm².

3. The electrode of claim 1 wherein the coating has surface features and the surface features vary in size.

4. The electrode of claim 1 wherein the coating comprises one of the group of tantalum, vanadium, titanium, molybdenum, hafnium, zirconium, niobium tungsten and platinum.

5. The electrode of claim 1 wherein the physical vapor deposition comprises one of the group of sputtering, cathodic arc deposition or thermal evaporation.

6. The electrode of claim 1 wherein the coating is applied to the body via one of a generally oblique coating flux or a low energy coating flux.

7. The electrode of claim 1 wherein the coating has pores.

8. The electrode of claim 7 further comprising a drug within the pores.

9. The electrode of claim 1 further comprising a second coating applied to the body.

10. The electrode of claim 9 wherein a second coating is applied directly to the body and the porous coating is applied to the second coating.

11. The electrode of claim 10 wherein the second coating protects the body from corrosion.

12. The electrode of claim 11 wherein the second coating in nonporous.

13. The electrode of claim 1 wherein the coating has a thickness between 1 and 100 micrometers.

14. The electrode of claim 1 wherein the coating has surface features having a size between approximately 10 and 1000 nanometers.

15. A process for making a high capacitance portion of an implantable electrode comprising the steps of:
maintaining a background pressure of gas in a sputter coating system containing at least one sputter target;
applying a voltage to the target to cause sputtering; and
sputtering for a period of time to produce a microscopically rough, metal coating on a portion of the electrode.

16. The process of claim 15 wherein the coating has surface features having a size between 10 nm and 1000 nm.

17. The process of claim 15 wherein the coating has surface features and the surface features vary in size.

18. The process of claim 15 wherein the coating comprises one of the groups of tantalum, vanadium, titanium, molybdenum, hafnium, zirconium, niobium, platinum and tungsten.

19. The process of claim 15 wherein the capacitance is between approximately 10 and 1000 milli Farads/cm².

20. The process of claim 15 wherein the coating is applied to the electrode via one of a generally oblique coating flux or a low energy coating flux.

21. The process of claim 15 further comprising a second coating applied to the electrode.

22. The process of claim 21 wherein the second coating is applied directly to the electrode.

23. The process of claim 22 wherein the second coating protects the electrode from corrosion.

24. The process of claim 22 wherein the second coating in nonporous.

25. The process of claim 15 wherein the coating has a thickness between 1 and 100 micrometers.

26. The process of claim 15 wherein the coating comprises at least one porous portion and at least one nonporous portion.

27. The process of claim 26 wherein the porous portion coats the nonporous portion.

28. The process of claim 15 wherein the coating has pores.

29. The process of claim 28 further comprising a drug within the pores.

30. An implantable medical device comprising:
a pacemaker having electrodes, the electrodes comprising:
a body; and
a biomedically compatible, microscopically rough, metal coating applied to at least a portion of the body via physical vapor deposition.
